(19) United States
(12) Patent Application Publication
(10) Pub. No.: US 2025/0014218 A1
(43) Pub. Date: Jan. 9, 2025

(54) Camera Probe Navigation within Cavity

(71) Applicant: University of Washington, Seattle, WA (US)

(72) Inventors: Eric J. SEIBEL, Seattle, WA (US); Chen GONG, Seattle, WA (US); Andrew LEWIS, Seattle, WA (US); Yaxuan ZHOU, Seattle, WA (US); Blake HANNAFORD, Seattle, WA (US)

(21) Appl. No.: 18/708,734

(22) PCT Filed: Nov. 9, 2022
(86) PCT No.: PCT/US2022/049443

§ 371 Date: May 9, 2024

Related U.S. Application Data
(60) Provisional application No. 63/277,928, filed on Nov. 10, 2021.

(51) Int. Cl. G06T 7/73; A61B 1/00; A61B 1/005; A61B 1/045; A61B 1/307; G06T 3/40; G06T 17/00; G06T 19/00; G06V 10/12; G06V 10/75; H04N 23/50; H04N 23/661

(52) U.S. Cl. CPC G06T 7/74; G06T 3/40; G06T 17/00; G06T 19/00; G06V 10/12; G06V 10/758; H04N 23/555; H04N 23/661; A61B 1/00009; A61B 1/00011; A61B 1/005; A61B 1/045; A61B 1/307; G06T 2207/10068

(57) ABSTRACT

A method includes capturing, via a camera that is inserted into a cavity, a first image of a surface defining the cavity and compressing the first image, using a compression algorithm, to generate a first feature vector. The method also includes identifying a second feature vector of a plurality of second feature vectors that best matches the first feature vector. The plurality of second feature vectors was generated by compressing second images of the surface using the compression algorithm. The second images were captured prior to insertion of the camera into the cavity and prior to capturing the first image. The method also includes generating, using the second feature vector, output indicating a position and/or an orientation of the camera as the camera captured the first image.

### CROSS-REFERENCE TO RELATED APPLICATION

This application claims priority to U.S. Provisional Patent Application No. 63/277,928, filed on Nov. 10, 2021, the entire contents of which are incorporated by reference herein.

### BACKGROUND

Flexible cystoscopy is a procedure that can be used for diagnosis and treatment of bladder cancer. Flexible cystoscopy involves insertion of a flexible catheter into a patient's bladder via the urethra so that a camera housed within the catheter can capture images of the interior surface of the bladder. A urologist can then use the images to diagnose the presence of bladder cancer or identify other abnormalities. Bladder cancer's high recurrence rate means that patients often return to urologists for follow-up cystoscopies several times per year for surveillance after initial diagnosis and treatment. However, many urologists practice in metropolitan areas, which can burden some patients living in more remote areas.

### SUMMARY

A first example is a method comprising: capturing, via a camera that is inserted into a cavity, a first image of a surface defining the cavity and a second image of the surface; identifying a first location of a feature in the first image and a second location of the feature in the second image using a scale-invariant feature transform algorithm; generating, using a structure from motion algorithm, a three-dimensional model of the surface using the first image, the first location, the second image, and the second location; compressing the first image and the second image, using a compression algorithm, to generate a first feature vector corresponding to the first image and a second feature vector corresponding to the second image; and generating an output indicating a first position and/or a first orientation of the camera as the camera captured the first image and a second position and/or a second orientation of the camera as the camera captured the second image.

A second example is a non-transitory computer readable medium storing instructions that, when executed by a probe system, cause the probe system to perform functions comprising: capturing, via a camera that is inserted into a cavity, a first image of a surface defining the cavity and a second image of the surface; identifying a first location of a feature in the first image and a second location of the feature in the second image using a scale-invariant feature transform algorithm; generating, using a structure from motion algorithm, a three-dimensional model of the surface using the first image, the first location, the second image, and the second location; compressing the first image and the second image, using a compression algorithm, to generate a first feature vector corresponding to the first image and a second feature vector corresponding to the second image; and generating an output indicating a first position and/or a first orientation of the camera as the camera captured the first image and a second position and/or a second orientation of the camera as the camera captured the second image.

A third example is a probe system comprising: a camera; one or more processors; and a computer readable medium storing instructions that, when executed by the one or more processors, cause the probe system to perform functions comprising: capturing, via the camera that is inserted into a cavity, a first image of a surface defining the cavity and a second image of the surface; identifying a first location of a feature in the first image and a second location of the feature in the second image using a scale-invariant feature transform algorithm; generating, using a structure from motion algorithm, a three-dimensional model of the surface using the first image, the first location, the second image, and the second location; compressing the first image and the second image, using a compression algorithm, to generate a first feature vector corresponding to the first image and a second feature vector corresponding to the second image; and generating an output indicating a first position and/or a first orientation of the camera as the camera captured the first image and a second position and/or a second orientation of the camera as the camera captured the second image.

A fourth example is a method comprising: capturing, via a camera that is inserted into a cavity, a first image of a surface defining the cavity; compressing the first image, using a compression algorithm, to generate a first feature vector; identifying a second feature vector of a plurality of second feature vectors that best matches the first feature vector, wherein the plurality of second feature vectors was generated by compressing second images of the surface using the compression algorithm, the second images being captured prior to insertion of the camera into the cavity and prior to capturing the first image; and generating, using the second feature vector, output indicating a position and/or an orientation of the camera as the camera captured the first image.

A fifth example is a non-transitory computer readable medium storing instructions that, when executed by a probe system, cause the probe system to perform functions comprising: capturing, via a camera that is inserted into a cavity, a first image of a surface defining the cavity; compressing the first image, using a compression algorithm, to generate a first feature vector; identifying a second feature vector of a plurality of second feature vectors that best matches the first feature vector, wherein the plurality of second feature vectors was generated by compressing second images of the surface using the compression algorithm, the second images being captured prior to insertion of the camera into the cavity and prior to capturing the first image; and generating, using the second feature vector, output indicating a position and/or an orientation of the camera as the camera captured the first image.

A sixth example is probe system comprising: a camera; one or more processors; and a computer readable medium storing instructions that, when executed by the one or more processors, cause the probe system to perform functions comprising: capturing, via the camera that is inserted into a cavity, a first image of a surface defining the cavity; compressing the first image, using a compression algorithm, to generate a first feature vector; identifying a second feature vector of a plurality of second feature vectors that best matches the first feature vector, wherein the plurality of second feature vectors was generated by compressing second images of the surface using the compression algorithm, the second images being captured prior to insertion of the camera into the cavity and prior to capturing the first image; and generating, using the second feature vector, output indicating a position and/or an orientation of the camera as the camera captured the first image.

When the term “substantially” or “about” is used herein, it is meant that the recited characteristic, parameter, or value need not be achieved exactly, but that deviations or variations, including, for example, tolerances, measurement error, measurement accuracy limitations, and other factors known to those of skill in the art may occur in amounts that do not preclude the effect the characteristic was intended to provide. In some examples disclosed herein, “substantially” or “about” means within +/−0-5% of the recited value.

The following document is incorporated by reference herein: Chen Gong, Yaxuan Zhou, Andrew Lewis, Pengcheng Chen, Jason R. Speich, Michael P. Porter, Blake Hannaford and Eric J. Seibel, “Real-Time Camera Localization during Robot-Assisted Telecystoscopy for Bladder Cancer Surveillance,” Journal of Medical Robotics Research Vol. 7, Nos. 2 and 3 (2022), pp. 2241002-1-2241002-17, https://doi.org/10.1142/S2424905X22410021.

These, as well as other aspects, advantages, and alternatives will become apparent to those of ordinary skill in the art by reading the following detailed description, with reference where appropriate to the accompanying drawings. Further, it should be understood that this summary and other descriptions and figures provided herein are intended to illustrate the invention by way of example only and, as such, that numerous variations are possible.

### BRIEF DESCRIPTION OF THE DRAWINGS

FIG. 1 is a block diagram of a probe system, according to an example.

FIG. 2 is a schematic diagram of a catheter and a camera operating within a cavity, according to an example.

FIG. 3 shows two images and two feature vectors, according to an example.

FIG. 4 shows a three-dimensional model of a surface, according to an example.

FIG. 5 shows two images and two feature vectors, according to an example.

FIG. 6 shows a three-dimensional model of a surface, according to an example.

FIG. 7 is a block diagram of a method, according to an example.

FIG. 8 is a block diagram of a method, according to an example.

### DETAILED DESCRIPTION

This disclosure includes examples that can facilitate more convenient treatment and monitoring procedures for bladder cancer patients, especially bladder cancer patients that live in remote areas that lack a large number of urologists available to perform conventional (e.g., manual) cystoscopy procedures. A conventional cystoscopy procedure usually involves a urologist manually operating a robotic catheter equipped with a camera at the tip of the catheter. That is, the catheter is inserted into the urethra of the patient and the urologist manipulates the catheter to move throughout the urinary tract and bladder, capturing images at areas of interest within the urinary tract and bladder.

Telecystoscopy is similar to a conventional cystoscopy procedure, except that telecystoscopy is performed remotely by a urologist that is not located in the same place as the patient under examination. For example, a urologist can use a joystick or a similar user interface to send commands over the internet to a robotic probe system hundreds of miles away. The probe system then receives the commands and responsively moves and orients its catheter and/or camera and otherwise operates as directed by the urologist's commands.

In another example, remote cystoscopy is similar to a conventional cystoscopy procedure, except that the cystoscopy procedure is performed by a physician assistant, a general practitioner, or a nurse remotely from a urologist. For example, the urologist may be “on call” for these remote cystoscopic examinations by a medical professional who is not highly trained for such a procedure. If there is a complication, the on-call urologist would be consulted and provide advice to the cystoscopic examiner.

In another example, the robotic probe operates in a more automated fashion. For instance, a nurse can set up the robotic probe by placing the tip of the catheter (e.g., the camera) near or just inside the patient's urethra, or just within the patient's bladder, with a predetermined position and/or orientation relative to the patient's body. Then, the nurse can press a button or otherwise use a user interface to instruct the robotic probe to move the catheter tip and camera along a predetermined trajectory, capturing images at predetermined waypoints within the bladder. These images can be inspected off site by a urologist well after the cystoscopy is performed.

An issue that can arise in either telecystoscopy, remote manual cystology, or in a robotically-assisted automated cystoscopy procedure is that it can be difficult to know exactly where the camera is viewing and located within the patient's bladder or urinary tract without the urologist using tactile feedback and expertise acquired by performing numerous manual cystoscopy procedures. As such, it can be difficult to correlate images captured by the camera with a particular location within the bladder which can be useful in determining that the entire bladder was imaged and the examination is completed.

Accordingly, a urologist can manually perform a patient's initial cystoscopy procedure and use the images captured during the initial cystoscopy procedure to generate a three-dimensional model of the patient's bladder. Perhaps months later, a telecystoscopy procedure, remote manual cystoscopy procedure, or an automated cystoscopy procedure can be performed on the patient, with the three-dimensional model of the patient's bladder generated via the initial cystoscopy procedure being used to guide or navigate the camera of the probe system.

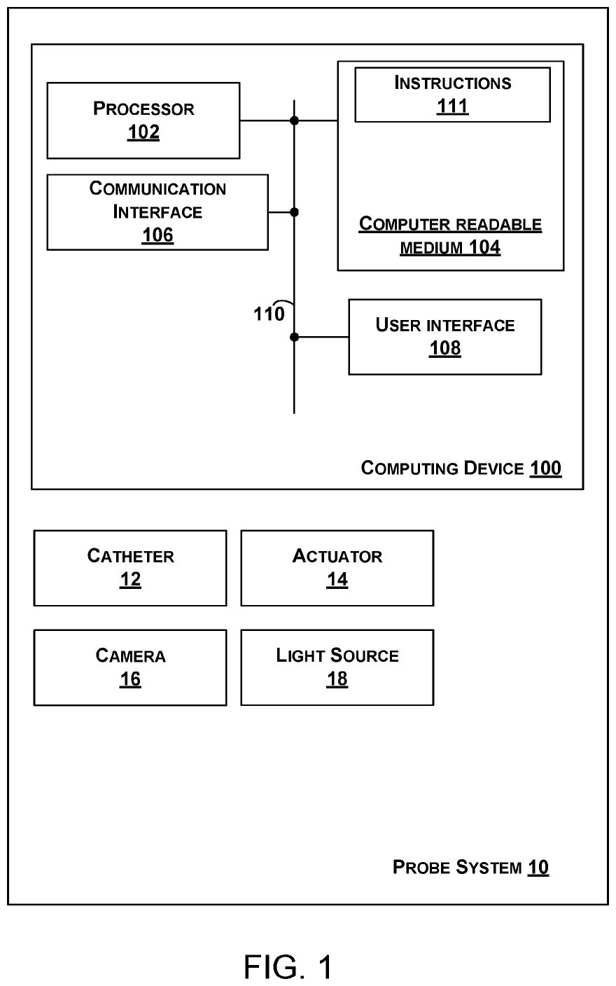

FIG. 1 is a block diagram of a probe system 10. The probe system 10 includes a catheter 12, an actuator 14, a camera 16, a light source 18, and a computing device 100.

The catheter 12 is a flexible tube that houses electrical wiring that provides power and control signals for the actuator 14 and/or the camera 16. Generally, the catheter 12 can be articulated to telescopically extend along a (e.g., longitudinal) axis of the catheter 12, to roll about the axis of the catheter 12, and/or to deflect relative to the axis of the catheter 12.

The actuator 14 can take the form of any electromechanical system configured to articulate the catheter 12 as discussed above.

The camera 16 can take the form of any imaging device configured to sense wavelengths of infrared, visible, and/or ultraviolet radiation. For example, the camera 16 can include an image sensor and/or focusing optics. The camera 16 is generally positioned at a distal end of the catheter 12. The lens system can vary the viewing angle from the longitudinal axis of the catheter from centered on axis to orthogonal to the axis.

The light source 18 can take the form of a light emitting diode (LED), but other examples are possible and this light can be brought to the distal end of the catheter by optical fibers. The light source 18 is configured to illuminate the field of view of the camera 16 so that images captured with the camera 16 have suitable brightness and/or contrast. The light source 18 is generally positioned at a distal end of the catheter 12 adjacent to the camera 16.

The computing device 100 includes one or more processors 102, a non-transitory computer readable medium 104, a communication interface 106, and a user interface 108. Components of the computing device 100 are linked together by a system bus, network, or other connection mechanism 110.

The one or more processors 102 can be any type of processor(s), such as a microprocessor, a field programmable gate array, a digital signal processor, a multicore processor, etc., coupled to the non-transitory computer readable medium 104.

The non-transitory computer readable medium 104 can be any type of memory, such as volatile memory like random access memory (RAM), dynamic random access memory (DRAM), static random access memory (SRAM), or non-volatile memory like read-only memory (ROM), flash memory, magnetic or optical disks, or compact-disc read-only memory (CD-ROM), among other devices used to store data or programs on a temporary or permanent basis.

Additionally, the non-transitory computer readable medium 104 can store instructions 111. The instructions 111 are executable by the one or more processors 102 to cause the computing device 100 (e.g., the probe system 10) to perform any of the functions or methods described herein.

The communication interface 106 can include hardware to enable communication within the computing device 100 and/or between the computing device 100 and one or more other devices. The hardware can include any type of input and/or output interfaces, a universal serial bus (USB), PCI Express, transmitters, receivers, and antennas, for example. The communication interface 106 can be configured to facilitate communication with one or more other devices, in accordance with one or more wired or wireless communication protocols. For example, the communication interface 106 can be configured to facilitate wireless data communication for the computing device 100 according to one or more wireless communication standards, such as one or more Institute of Electrical and Electronics Engineers (IEEE) 801.11 standards, ZigBee standards, Bluetooth standards, etc. As another example, the communication interface 106 can be configured to facilitate wired data communication with one or more other devices. The communication interface 106 can also include analog-to-digital converters (ADCs) or digital-to-analog converters (DACs) that the computing device 100 can use to control various components of the computing device 100 or external devices.

The user interface 108 can include any type of display component configured to display data. As one example, the user interface 108 can include a touchscreen display. As another example, the user interface 108 can include a flat-panel display, such as a liquid-crystal display (LCD) or a light-emitting diode (LED) display. The user interface 108 can include one or more pieces of hardware used to provide data and control signals to the computing device 100. For instance, the user interface 108 can include a mouse or a pointing device, a keyboard or a keypad, a microphone, a touchpad, or a touchscreen, among other possible types of user input devices. Generally, the user interface 108 can enable an operator to interact with a graphical user interface (GUI) provided by the computing device 100 (e.g., displayed by the user interface 108).

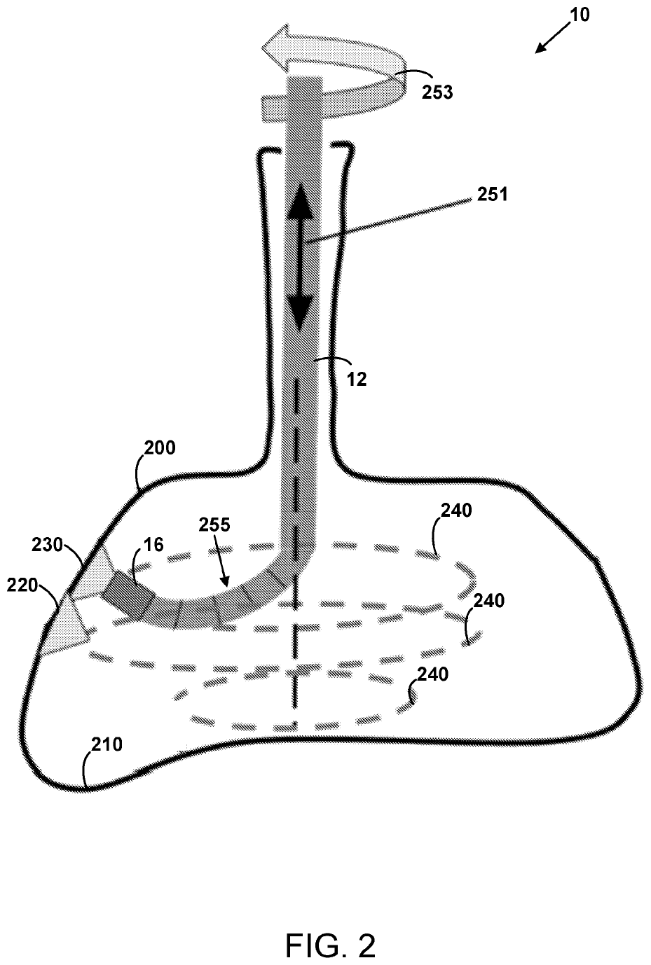

FIG. 2 is a schematic diagram of the catheter 12 and the camera 16 operating within a cavity 200. The cavity 200 is typically a urinary tract and/or a bladder but can also take the form of a stomach, a vagina, a, a uterus, a ureter, a kidney chamber, a heart chamber, a brain ventricle, a gastrointestinal tract, a nasal cavity, or a lung. Other examples are possible. In non-medical applications, the cavity 200 can take the form of a pipe, a storage tank, a cave, a room within a building, a cavity within an aircraft engine or within an aircraft wing, or a ceiling plenum space. Generally, the cavity 200 can take the form of any enclosed space under examination by the probe system 10.

During an initial examination of the cavity 200 (e.g., a bladder), a urologist can use the probe system 10 to generate a library of feature vectors and a three-dimensional model of a surface 210 of the cavity 200, as described below. The feature vectors and the model can be used during a subsequent examination of the cavity 200 for the purpose of navigating the catheter 12 and/or the camera 16 within the same cavity 200. The subsequent examination of the cavity 200 could be a telecystoscopy procedure, remote manual cystoscopy, or an automated cystoscopy procedure, for example, whereas the initial examination could be a manual cystoscopy procedure performed by a urologist.

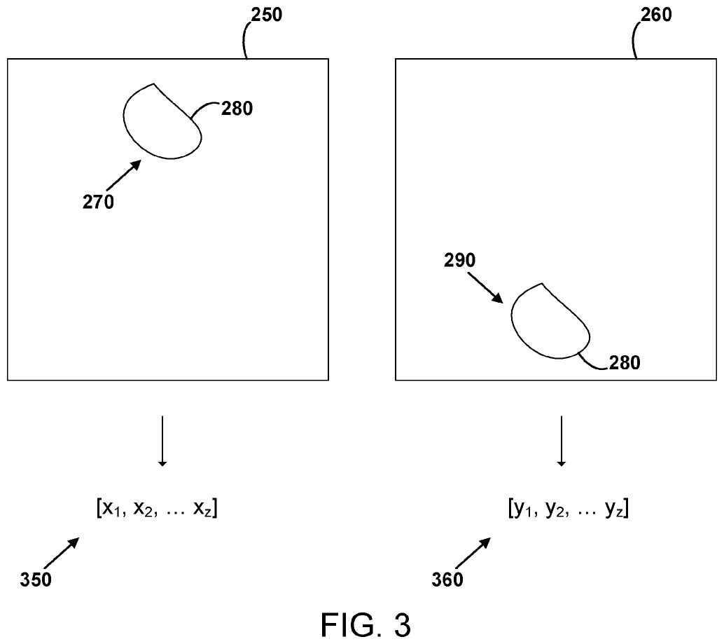

During an initial examination, the probe system 10 can capture, via the camera 16 that is inserted into the cavity 200 via the catheter 12, a first image of the surface 210 defining the cavity 200 and a second image of the surface 210. For instance, the camera 16 can capture an image 250 of a location 220 on the surface 210 and an image 260 of a location 230 on the surface 210. (The image 250 and the image 260 are shown in FIG. 3.) Generally, the probe system 10 uses the camera 16 to capture tens, hundreds, or thousands of images of the surface 210. For example, the probe system 10 can capture the images of the surface 210 such that every location of interest on the surface 210 is included in at least two of the images captured by the camera 16. The probe system 10 can capture the images in an order that corresponds with a trajectory 240 of the camera 16. The trajectory 240 can be defined by a sequence of positions and orientations of the camera 16 defined by a degree of extension 251 of the catheter 12, a roll angle 253 of the catheter 12, and/or a deflection angle 255 of the catheter 12.

FIG. 3 shows the image 250 corresponding to the location 220 and the image 260 corresponding to the location 230. The probe system 10 or another computing system identifies a location 270 of a feature 280 in the image 250 and a location 290 of the feature 280 in the image 260 using a scale-invariant feature transform (SIFT) algorithm. The location 270 and the location 290 could be defined by Cartesian coordinates within the image 250 and the image 260, respectively. The feature 280 could be a lesion, but other examples are possible. The location 270 is higher within the image 250 than the location 290 is within the image 260 because the location 220 is lower than the location 230. Generally, the probe system 10 or another computing system identifies many features that are common to sets of two more images of the surface 210 captured by the camera 16.

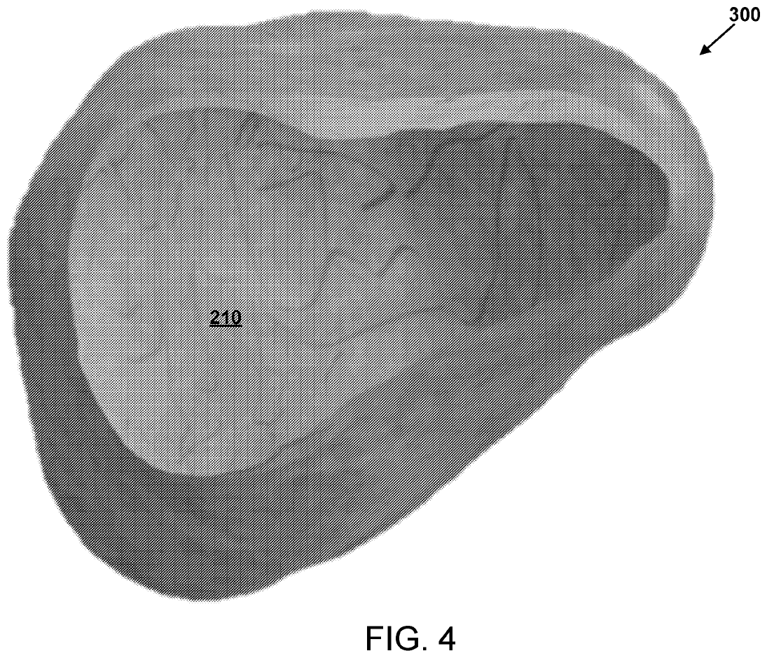

As shown in FIG. 4, the probe system 10 or another computing system generates, using a structure from motion (SfM) algorithm, a three-dimensional model 300 of the surface 210 using the image 250, the location 270, the image 260, and the location 290. More particularly, the probe system 10 or another computing system uses the location 270 of the feature 280 within the image 250, the known position and orientation of the camera 16 when the camera captured the image 250, the location 290 of the feature 280 within the image 260, and the known position and orientation of the camera 16 when the camera captured the image 260 to construct the three-dimensional model 300.

More particularly, the probe system 10 or another computing system uses the SfM algorithm to generate numerous coordinates for a point cloud model of the surface 210 using numerous images of the surface 210. Various images captured by the camera 16 contain overlapping features or sections of the surface 210. The SfM algorithm uses this overlap between the images, the relative locations of common features within the images, and the known position and orientation of the camera 16 when the camera 16 captured each image, to generate the three-dimensional model 300.

For example, the probe system 10 or another computing system can generate the three-dimensional model 300 using a Poisson surface reconstruction algorithm. Additionally or alternatively, the probe system 10 or another computing system can generate the three-dimensional model 300 such that a texture map defined by the image 250 and a texture map defined by the image 260 are projected onto the three-dimensional model 300 of the surface 210. The three-dimensional model 300 can be used to locate and help navigate the catheter 12 and/or the camera 16 in future examination procedures.

Referring back to FIG. 3, the probe system 10 or another computing system compresses the image 250 and the image 260, using a compression algorithm such as dimensional reduction and/or singular value decomposition, to generate a feature vector 350 corresponding to the image 250 and a feature vector 360 corresponding to the image 260. That is, the compression algorithm compresses the image 250 and the image 260 into respective z-dimension vectors. For example, the image 250 could be a 10 megapixel monochrome image and z could be equal to 20, but other examples are possible. Generally the probe system 10 or another computing system uses the compression algorithm to compress many images captured by the camera 16 while the camera 16 transits the trajectory 240. The probe system 10 or another computing system can select dimensions of the feature vector 350 and the feature vector 360 using principal component analysis, for example.

Performing dimensional reduction upon the image 250 and the image 260 can involve transforming the image 250 into the feature vector 350 having the dimensions selected using principal component analysis and transforming the image 260 into the feature vector 360 having the dimensions selected using principal component analysis.

Generally, the feature vector 350, the feature vector 360, and any feature vector corresponding to an image captured by the camera 16 and used to generate the three-dimensional model 300 can be used to locate and help navigate the catheter 12 and/or the camera 16 in future examination procedures, as described below.

Additionally, the probe system 10 or another computing system generates an output indicating a position and/or an orientation of the camera 16 as the camera 16 captured the image 250 and a position and/or an orientation of the camera 16 as the camera 16 captured the image 260. More generally, the probe system 10 or another computing system stores and/or displays data that correlates each captured image with a position and an orientation of the camera 16 that corresponds to that particular image. The output can be used to navigate the catheter 12 and/or the camera 16 in future examination procedures, as described below.

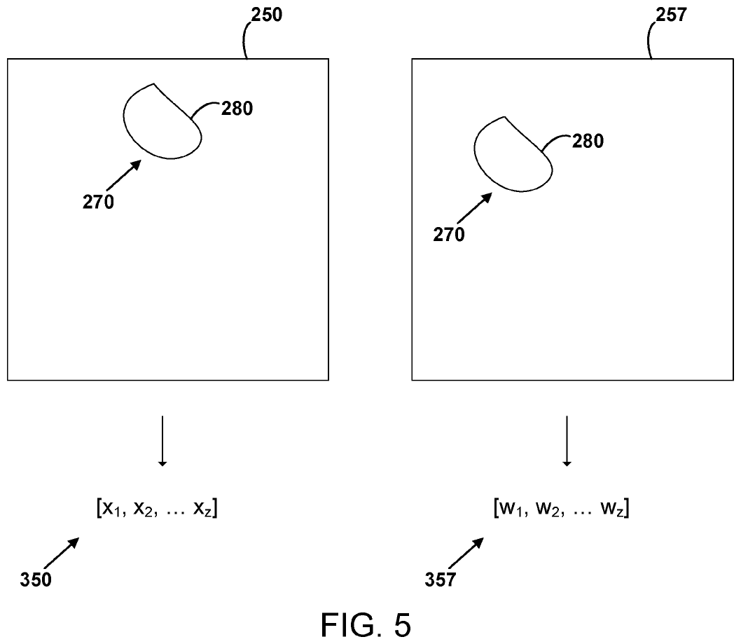

The probe system 10 can also be used to perform a subsequent cystoscopy procedure, perhaps weeks or months after the initial procedure described above. Referring to FIG. 5, the probe system 10 captures, via the camera 16 inserted into the cavity 200, an image 257 of the surface 210 defining the cavity 200. The image 257 can roughly correspond to the location 220 shown in FIG. 2 and the image 250, although that may be initially unknown due to uncertainty about the position and/or orientation of the camera 16 while capturing the image 257.

Next, the probe system 10 or another computing system compresses the image 257, using the same compression algorithm used to generate the feature vector 350 from the image 250, to generate a feature vector 357.

The probe system 10 or another computing system then identifies the feature vector 350, from among many feature vectors stored in a database, as best matching the feature vector 357. The feature vectors stored in the database were generated by compressing images of the surface 210 using the compression algorithm, with the images being captured prior to insertion of the camera 16 (e.g., for the subsequent cystoscopy procedure) into the cavity 200 and thus prior to capturing the image 257. More specifically, the feature vectors stored in the database generally correspond to images captured during the initial cystoscopy procedure discussed above.

Identifying the feature vector 350 as the best match for the feature vector 357 typically involves determining that a Euclidean distance between the feature vector 357 and the feature vector 350 is less than any Euclidean distance between the feature vector 357 and other feature vectors stored in the database.

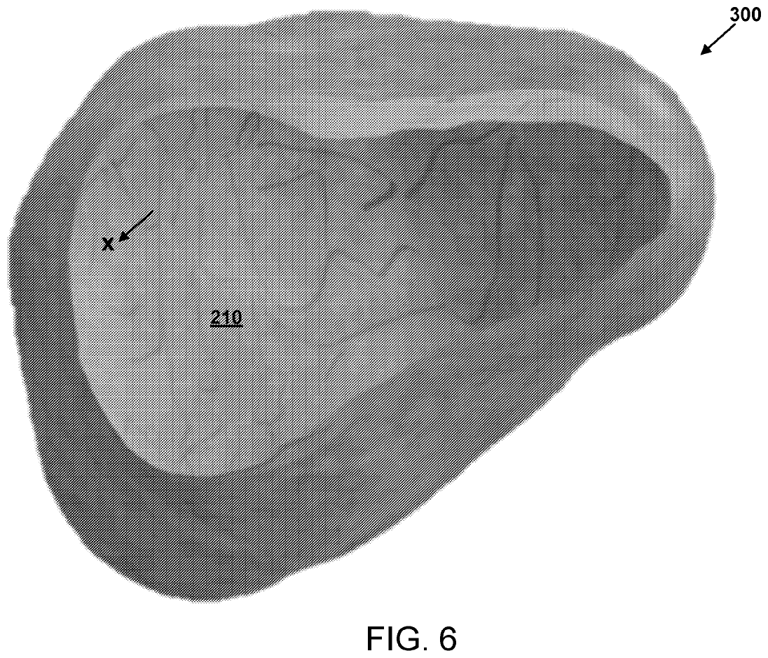

After the feature vector 350 has been identified, the probe system 10 or another computing system generates, using the feature vector 350, output indicating a position and/or an orientation of the camera 16 as the camera 16 captured the image 257. For example, the probe system 10 can store or display data indicating the position and/or the orientation the camera 16 had when the camera 16 captured the image 257, shown as an example in the form of an ‘x’ in FIG. 6. More particularly, the probe system 10 or another computing system could graphically indicate the position and/or the orientation of the camera 16 relative to the three-dimensional model 300 of the surface 210. Using the probe system 10 to graphically indicate the position and/or the orientation of the camera 16 relative to the three-dimensional model 300 of the surface 210 could be useful in remote manual cystology in that a local human user could use the displayed information to inform manual control of the catheter 12.

In the telecystoscopy context, the operator that is remote from the patient could also inform control of the catheter 12 using the output that is graphically displayed by a computing device that is local to the operator. Thus, in some examples, the output representing the position and/or the orientation of the camera 16 is transmitted by the probe system 10 local to the patient via a telecommunications network to a computing device local to the operator.

In the fully automated context, the probe system 10 can automatically use the (e.g., stored) output indicating the position and/or the orientation of the camera 16 as feedback to further control the position and/or orientation of the catheter 12. As such, the probe system 10 can determine, based on the output indicating the position and/or the orientation of the camera 16, an action that includes extending, retracting, rolling, or deflecting a catheter that houses the camera 16. The probe system 10 accordingly can cause the catheter 12 to perform the determined action.

In some examples, the probe system 10 or another computing system will simply infer that the position and/or orientation of the camera used to capture the image 250 and the position and/or orientation of the camera 16 used to capture the image 257 are the same, and generate the output accordingly.

In other examples, the probe system 10 or another computing system can account for the image 257 and the image 250 not being perfect matches and therefore not having the exact same camera position and/or orientation. Accordingly, the probe system 10 or another computing system can determine the position and/or the orientation of the camera 16 while capturing the image 257 by applying a transfer function to the image 257. The transfer function maps the image 250 to the three-dimensional model 300 of the surface 210. As such, the probe system 10 or another computing system can generate the output as indicating the position and/or the orientation as provided by applying the transfer function to the 257 image.

As noted above, the subsequent cystoscopy procedure can take the form of a telecystoscopy procedure. As such, the probe system 10 can receive, via a telecommunications network, a command for the camera 16 to move such that the camera 16 has a particular position and the orientation. Next, the probe system 10 adjusts, in response to receiving the command, the camera 16 such that the camera 16 has the position and the orientation specified by the command. In this context, capturing the image 257 can include capturing the image 257 while the camera 165 has the position and the orientation specified by the command.

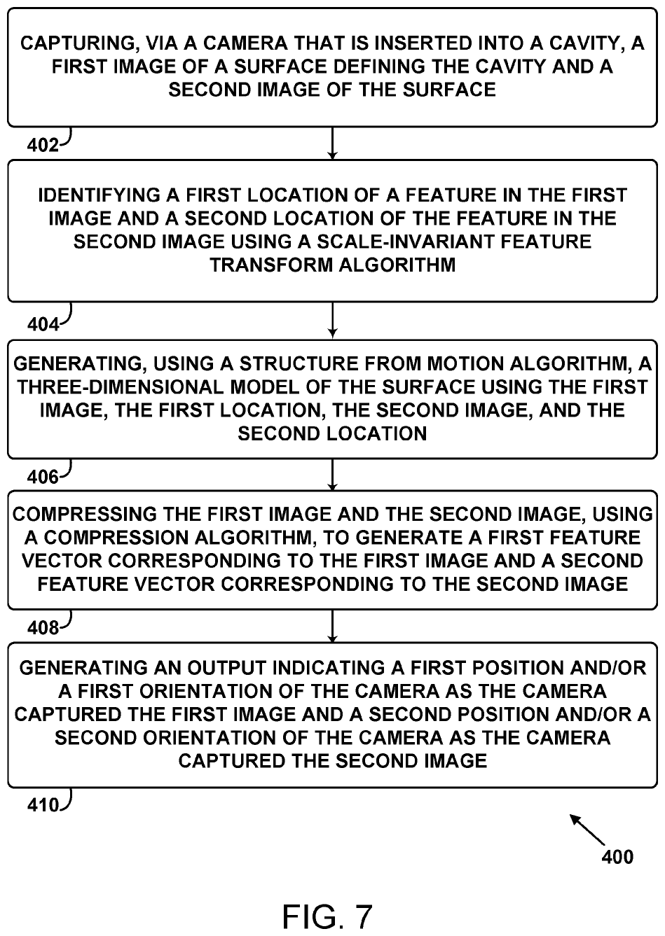

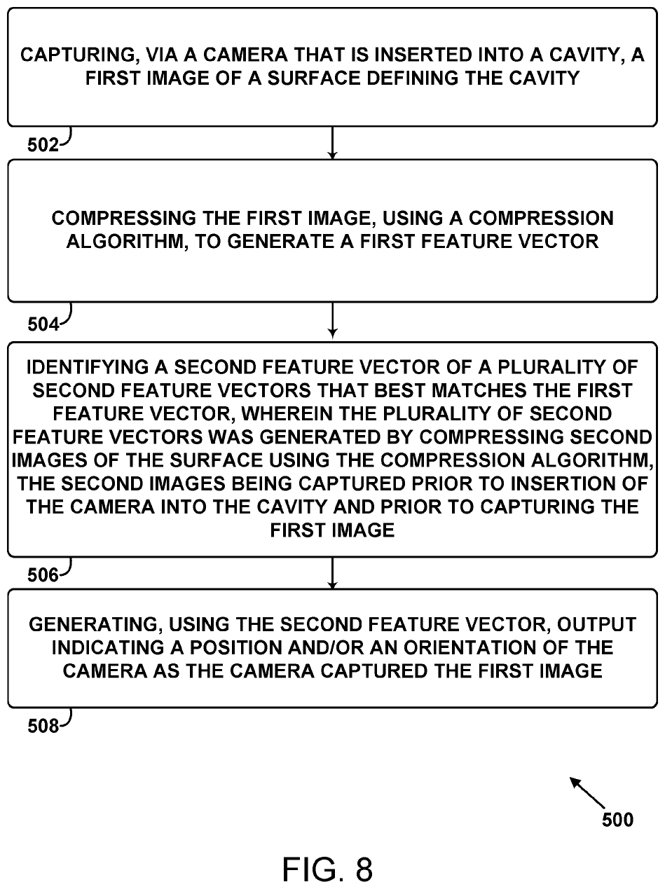

FIG. 7 and FIG. 8 are block diagrams of a method 400 and a method 500, which in some examples are methods of operating the probe system 10. As shown in FIG. 7 and FIG. 8, the method 400 and the method 500 include one or more operations, functions, or actions as illustrated by blocks 402, 404, 406, 408, 410, 502, 504, 506, and 508. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 402, the method 400 includes capturing, via the camera 16 that is inserted into the cavity 200, the image 250 of the surface 210 defining the cavity 200 and the image 260 of the surface 210. Functionality related to block 402 is described above with reference to FIG. 2 and FIG. 3.

At block 404, the method 400 includes identifying the location 270 of the feature 280 in the image 250 and the location 290 of the feature 280 in the image 260 using the scale-invariant feature transform algorithm. Functionality related to block 404 is described above with reference to FIG. 2 and FIG. 3.

At block 406, the method 400 includes generating, using the structure from motion algorithm, the three-dimensional model 300 of the surface 210 using the image 250, the location 270, the image 260, and the location 290. Functionality related to block 406 is described above with reference to FIG. 3 and FIG. 4.

At block 408, the method 400 includes compressing the image 250 and the image 260, using the compression algorithm, to generate a feature vector 350 corresponding to the image 250 and the feature vector 360 corresponding to the image 260. Functionality related to block 408 is described above with reference to FIG. 3.

At block 410, the method 400 includes generating the output indicating the position and/or the orientation of the camera 16 as the camera 16 captured the image 250 and the position and/or the orientation of the camera 16 as the camera 16 captured the image 260. Functionality related to block 410 is described above with reference to FIGS. 2-4.

At block 502, the method 500 includes capturing, via the camera 16 that is inserted into the cavity 200, the image 257 of the surface 210 defining the cavity 200. Functionality related to block 502 is described above with reference to FIG. 5.

At block 504, the method 500 includes compressing the image 257, using the compression algorithm, to generate the feature vector 357. Functionality related to block 504 is described above with reference to FIG. 5.

At block 506, the method 500 includes identifying the feature vector 350 of the plurality of feature vectors that best matches the feature vector 357. The plurality of feature vectors was generated by compressing images of the surface 210 using the compression algorithm, and the images were captured prior to insertion of the camera 16 into the cavity 200 and prior to capturing the image 257. Functionality related to block 506 is described above with reference to FIG. 5.

At block 508, the method 500 includes generating, using the feature vector 350, output indicating the position and/or the orientation of the camera 16 as the camera 16 captured the image 257. Functionality related to block 508 is described above with reference to FIG. 6.

While various example aspects and example embodiments have been disclosed herein, other aspects and embodiments will be apparent to those skilled in the art. The various example aspects and example embodiments disclosed herein are for purposes of illustration and are not intended to be limiting, with the true scope and spirit being indicated by the following claims.

1-10. (canceled)

11. A method comprising:
capturing, via a camera that is inserted into a cavity, a first image of a surface defining the cavity;
compressing the first image, using a compression algorithm, to generate a first feature vector;
identifying a second feature vector of a plurality of second feature vectors that best matches the first feature vector, wherein the plurality of second feature vectors was generated by compressing second images of the surface using the compression algorithm, the second images being captured prior to insertion of the camera into the cavity and prior to capturing the first image; and
generating, using the second feature vector, output indicating a position and/or an orientation of the camera as the camera captured the first image.

12. The method of claim 11, wherein the cavity comprises a stomach, a vagina, a bladder, a urinary tract, a uterus, a ureter, a kidney chamber, a heart chamber, a brain ventricle, a gastrointestinal tract, a nasal cavity, a lung, a pipe, a storage tank, a cave, a room within a building, a cavity within an aircraft engine or within an aircraft wing, or a ceiling plenum space.

13. The method of claim 11, wherein the camera is integrated into an articulating catheter configured for extension along an axis, roll about the axis, and deflection relative to the axis.

14. The method of claim 11, wherein compressing the first image using the compression algorithm comprises performing dimensional reduction upon the first image.

15. The method of claim 14, wherein performing dimensional reduction upon the first image comprises transforming the first image into the first feature vector having dimensions selected by performing principal component analysis on the second images.

16. The method of claim 15, wherein performing dimensional reduction upon the first image further comprises performing singular value decomposition upon the first image.

17. The method of claim 11, wherein identifying the second feature vector comprises determining that a Euclidean distance between the first feature vector and the second feature vector is smaller than any Euclidean distance between the first feature vector and other second feature vectors of the plurality of second feature vectors.

18. The method of claim 11, wherein generating the output comprises graphically indicating the position and/or the orientation of the camera relative to a three-dimensional model of the surface generated using the second images.

19. The method of claim 11, wherein the camera is a first camera, the position is a first position, the orientation is a first orientation, and the second images comprise a second image that corresponds to the second feature vector,
wherein generating the output comprises generating the output such that the output indicates the first position that is equal to a second position of a second camera as the second camera captured the second image and indicates the first orientation that is equal to a second orientation of the second camera as the second camera captured the second image.

20. The method of claim 11, wherein the second images comprise a second image that corresponds to the second feature vector, the method further comprising:
determining the position and/or the orientation by applying a transfer function to the first image, wherein the transfer function maps the second image to a three-dimensional model of the surface,
wherein generating the output comprises generating the output such that the output indicates the position and/or the orientation as provided by applying the transfer function to the first image.

21. The method of claim 11, further comprising:
receiving, via a telecommunications network, a command for the camera to move such that the camera has the position and the orientation; and
adjusting, in response to receiving the command, the camera such that the camera has the position and the orientation,
wherein capturing the first image comprises capturing the first image while the camera has the position and the orientation.

22. The method of claim 11, further comprising transmitting the output via a telecommunications network.

23. The method of claim 11, further comprising:
determining, based on the output, an action that includes extending, retracting, rolling, or deflecting a catheter that houses the camera; and
causing the catheter to perform the action.

24. The method of claim 11, wherein generating the output comprises storing the output.

25. A non-transitory computer readable medium storing instructions that, when executed by a probe system, cause the probe system to perform functions comprising:
capturing, via a camera that is inserted into a cavity, a first image of a surface defining the cavity;
compressing the first image, using a compression algorithm, to generate a first feature vector;
identifying a second feature vector of a plurality of second feature vectors that best matches the first feature vector, wherein the plurality of second feature vectors was generated by compressing second images of the surface using the compression algorithm, the second images being captured prior to insertion of the camera into the cavity and prior to capturing the first image; and
generating, using the second feature vector, output indicating a position and/or an orientation of the camera as the camera captured the first image.

26. A probe system comprising:
an articulating catheter configured for extension along an axis, roll about the axis, and deflection relative to the axis;
a camera that is integrated into the articulating catheter;
one or more processors; and
a computer readable medium storing instructions that, when executed by the one or more processors, cause the probe system to perform functions comprising:
capturing, via a camera that is inserted into a cavity, a first image of a surface defining the cavity;
compressing the first image, using a compression algorithm, to generate a first feature vector;
identifying a second feature vector of a plurality of second feature vectors that best matches the first feature vector, wherein the plurality of second feature vectors was generated by compressing second images of the surface using the compression algorithm, the second images being captured prior to insertion of the camera into the cavity and prior to capturing the first image; and
generating, using the second feature vector, output indicating a position and/or an orientation of the camera as the camera captured the first image.

27. The non-transitory computer readable medium of claim 25, wherein generating the output comprises graphically indicating the position and/or the orientation of the camera relative to a three-dimensional model of the surface generated using the second images.

28. The probe system of claim 26, wherein generating the output comprises graphically indicating the position and/or the orientation of the camera relative to a three-dimensional model of the surface generated using the second images.

29. The non-transitory computer readable medium of claim 25, the functions further comprising transmitting the output via a telecommunications network.

30. The probe system of claim 26, the functions further comprising transmitting the output via a telecommunications network.
